(19) United States
(12) Patent Application Publication
(10) Pub. No.: US 2008/0066321 A1
(43) Pub. Date: Mar. 20, 2008

(54) FINGER-MOUNTED SUTURE CUTTER

(76) Inventor: ROMMEL P. VALLERO, Davis, CA (US)

Correspondence Address: Robert Platt Bell;Registered Patent Attorney, P.O. Box 310, Aurora, NY, 13026, US

(21) Appl. No.: 11/855,222

(22) Filed: Sep. 14, 2007

Related U.S. Application Data
(60) Provisional application No. 60/846,047, filed on Sep. 20, 2006.

(51) Int. Cl. B26D 1/01; A61B 17/32

(52) U.S. Cl. 30/298; 606/174

(57) ABSTRACT

The present invention comprises a sterilized disposable suture cutter worn preferably on the small finger of the same hand used to hold the needle holder. The device can cut by pulling the cutting edge against a somewhat taut suture material in a slicing fashion. Alternatively, the small finger can be flexed causing the cutting edge to be drawn against an opposing flat surface in a chopping motion. This second chopping method would be useful in cutting sutures where it would be difficult or impossible to place tension onto the suture. When not being used, the device does not interfere with the normal motions necessary to place sutures.

### CROSS-REFERENCE TO RELATED APPLICATIONS

The present application is a Non-Provisional of Provisional U.S. Patent Application Ser. No. 60/846,047 filed on Sep. 20, 2006 and incorporated herein by reference in its entirety.

### FIELD OF THE INVENTION

The present invention relates to surgical instruments, in particular, to hand held devices used for suture cutting after a surgical knot is made.

### BACKGROUND OF THE INVENTION

Surgical activities involving the stitching of tissue require that the suture be severed when a stitch has been completed. This usually requires that the operator release his/her grasp of the tool used to hold the needle, place it down somewhere within the sterile field, pick up a pair of scissors to make the cut, place down the scissors in the sterile field and re-grasp the needle holder in order to place another stitch.

Because most suturing procedures require multiple stitches and multiple suture cuts, it would be desirable in situations such as those described above if the cutting could be made without the need to release the instrument already being held.

Prior art related to this matter have tried to address some of these issues, but have certain drawbacks. The surgical cutting instrument as described by Gibbens (U.S. Pat. No. 4,726,371) is a finger-mounted device that uses a miniature scissors apparatus to cut suture without having to release an instrument already being held. Gibbens requires that the operator oppose the fingers to depress the lever in order to make a cut. This hand motion is somewhat difficult to do, especially if multiple cuts need to be made in succession. The muscles used to oppose the fingers in this manner are inherently weaker than muscles used to flex and extend the fingers and can tire easily. The sharp edges and points of the scissors are also exposed and increase the risk of operator or patient injury should there be unwanted or unexpected movement during the suturing process.

The combined thimble and cutter invented by Delinanos (U.S. Pat. No. 2,704,889) was intended to cut thread with a fixed blade screwed into a sewing thimble. Although this device allows cutting of thread and other similar material without any complicated movements of the finger, the cutting blade is tucked away and close to the thimble and lacks the ability to guide the thread or suture towards the cutting edge. This hinders the invention's ability to make quick easy cuts through thread or other similar material. Delinanos' thimble mounted invention also lacks the ability to adjust to different sized fingers, which also limits its use.

The suture cutter described by Swor (U.S. Pat. No. 4,969,893) uses a fixed blade cutter on a device worn on the wrist. This embodiment guides the suture towards the cutting edge, but does not allow the edge to get close to the suture knot and requires the use of larger, less dexterous muscles to make the cut.

The finger mounted suture cutters described by Miklos (Pub. No. US 2004/0231167 A1) and Shinichi (Patent No. JP2005111198) both have designs which allows for the guiding of suture into the blade. However, Miklos's invention requires that the operator use a jabbing or stabbing motion to cut the suture instead of a more fluid and controlled flexion or extension of the finger. Both Miklos's and Shinichi's cutters are also placed close to the finger and limits the ability to cut sutures very short as is sometimes desired, or to enable cutting in tight spaces like in the mouth or between fingers and toes. These cutters also lack the ability to cut material unless it is pulled taut and drawn against the cutting blade.

### SUMMARY OF THE INVENTION

It is an object of the present invention to address the shortcomings of the prior art and to provide a cutting instrument that minimizes the problems described above. It is also an object of the present invention to provide a quicker and safer method of cutting suture and other like material.

The present invention comprises a sterilized disposable suture cutter worn preferably on the small finger of the same hand used to hold the needle holder. The device can cut by pulling the cutting edge against a somewhat taut suture material in a slicing fashion. Alternatively, the small finger can be flexed causing the cutting edge to be drawn against an opposing flat surface in a chopping motion. This second chopping method would be useful in cutting sutures where it would be difficult or impossible to place tension onto the suture. When not being used, the device does not interfere with the normal motions necessary to place sutures.

### BRIEF DESCRIPTION OF THE DRAWINGS

FIG. 1 is a perspective view of a first embodiment of the present invention.

FIG. 2 is a side view of a first embodiment of the present invention showing how the suture cutter moves when the finger is flexed.

### DETAILED DESCRIPTION OF THE INVENTION

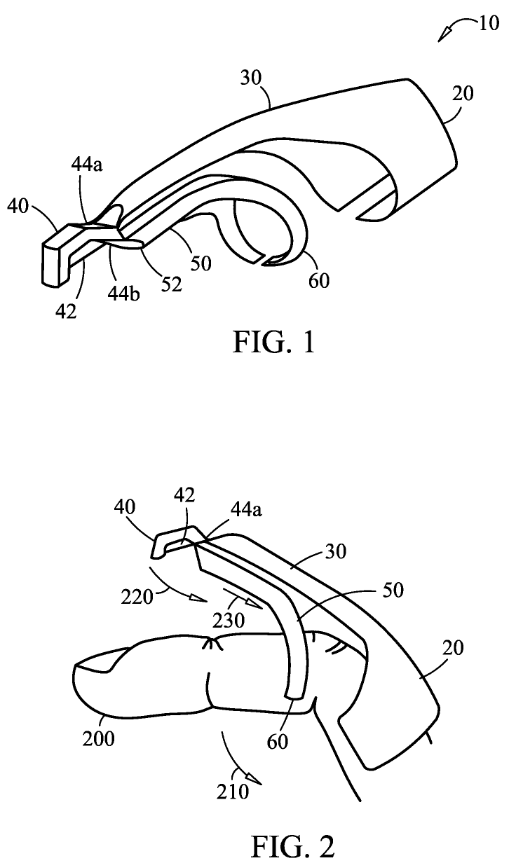

Referring to FIG. 1, the apparatus of the present invention comprises preferably a molded plastic unit 10 with an adjustable band 20 on one end designed to fit over variable finger sizes and a protruding curved arm 30 on the other end. The extended curved arm 30 allows the operator to reach into tight spaces and allows for close and precise placement of blade 42 onto a suture. At the end of the curved arm 30 is a pivoting head 40 in which a razor sharp cutting blade 42 is enclosed.

Referring to FIG. 2, blade 42 is recessed back to prevent contact with anything except a suture. Head 40 may move in a semi circular arc due to two thin, flexible plastic hinges 44a, 44b connected to the curved arm 30 and a lower arm 50. Lower arm 50 fits over a more distal portion of the finger 200 with a similar adjustable band 60 to fit variable finger sizes. Pivoting head 40 can be made to pull the cutting edge 42 against an opposing flat cutting surface 52 by flexing finger 200 and pulling on the lower arm as illustrated by arrows 210, 220, and 230 in FIG. 2. Preferably the apparatus may be used on the small (pinky) finger, but it may also be made in other sizes to be used on other fingers and for persons having different sized fingers.

In the finger mounted cutter of the present invention, the hinge portions 44a and 44b may comprise a live hinge of continuous flexible material (e.g., plastic), continuous with substantially all of the rest of the finger mounted cutter (e.g., other than the cutting blade 42) in order to reduce assembly steps needed for manufacture. The device may also utilize other types of hinges as well, within the spirit and scope of the present invention. Similarly cutting blade 42 may be formed of the same plastic material as the rest of the device, or may comprise a blade insert (e.g., metal blade) molded or inserted into head 40.

Other possible uses and variations are possible within the spirit and scope of the present invention. The suture cutter of the present invention has been designed to assist medical practitioners in the process of suturing tissue together. Because it allows for the cutting of sutures without having to constantly change instruments at the end of each suture placement, it provides for a quicker and safer alternative to the Prior Art. However, it may also be employed by individuals wishing to cut string, yarn, fishing line, and the like, with a small, safe, readily available instrument. The cutter also speeds the process of wrapping gifts when it is used to cut tape, ribbon, and gift wrapping paper. Envelopes can also be opened with the cutter, a safer alternative to using a sharp tipped letter opener.

In the preferred embodiment described above, the suture cutter may be packaged in a sterile fashion and disposed after one use. However, the suture cutter could also be created from other materials such as metal and re-used and sterilized. The entire unit may be made sterile along with other re-useable surgical instruments. The entire unit may be molded from a single piece of plastic for low cost and to be made disposable. A blade insert 42 may be molded into the plastic portion forming head 40, or alternately, a sharp portion of plastic may be used. The unit may also be made of metal or other materials if, for example, it is desired to reuse the device.

While the preferred embodiment and various alternative embodiments of the invention have been disclosed and described in detail herein, it may be apparent to those skilled in the art that various changes in form and detail may be made therein without departing from the spirit and scope thereof.

I claim:

1. A finger-mounted cutter, comprising:
a first arm adapted to couple to a first portion of a human finger;
a second arm adapted to couple to a second portion of a human finger;
a pivoting head, hinged to the first arm through a hinged portion and coupled to the second arm; and
a cutting blade mounted in the pivoting head,
wherein when the human finger is flexed, the second arm pulls the pivoting head causing the pivoting head to pivot along an axis of the hinged portion to cause the cutting blade to move into position for cutting.

2. The finger-mounted cutter of claim 1, further comprising:
a cutting block, mounted to or part of the second arm,
wherein when the pivoting head pivots along the axis of the hinged portion, the cutting blade comes into contact with the cutting block.

3. The finger mounted cutter of claim 2, wherein the first arm include an adjustable finger mount to accommodate different finger diameters.

4. The finger mounted cutter of claim 3, wherein the second arm include an adjustable finger mount to accommodate different finger diameters.

5. The finger mounted cutter of claim 4, wherein the adjustable finger mounts of the first arm and the second arm comprise flexible portions of the first arm and second arm having sufficient spring tension to attach to the first and second portions of a human finger so as to securely clip the finger mounted cutter to the first and section portions of the human finger and to accommodate different finger diameters.

6. The finger mounted cutter of claim 1, wherein the cutting blade is at least partially enclosed by the pivoting head so as to prevent the inadvertent cutting of surrounding objects or material and a portion of the sharp edge remains exposed to allow cutting by drawing the edge against the material to be cut.

7. The finger mounted cutter of claim 2, wherein when the pivoting head pivots along the axis of the hinged portion, the cutting blade comes into contact with the cutting block to cut material.

8. The finger mounted cutter of claim 1, wherein the first hinge portion comprises a live hinge of continuous flexible material, continuous with substantially all of the rest of the finger mounted cutter in order to reduce assembly steps needed for manufacture.

9. The finger mounted cutter of claim 1, wherein the a pivoting head, is coupled to the second arm through a second hinge portion, such that when the human finger is flexed, the second arm pulls the pivoting head toward the human finger.

10. The finger mounted cutter of claim 9, wherein the second hinge portion comprises a live hinge of continuous flexible material, continuous with substantially all of the rest of the finger mounted cutter in order to reduce assembly steps needed for manufacture.

11. A finger-mounted suture cutter, comprising:
a first arm adapted to couple to a first portion of a human finger;
a second arm adapted to couple to a second portion of a human finger;
a pivoting head, hinged to the first arm through a hinged portion and coupled to the second arm; and
a cutting blade mounted in the pivoting head,
wherein when the human finger is flexed, the second arm pulls the pivoting head causing the pivoting head to pivot along an axis of the hinged portion to cause the cutting blade to move into position for cutting sutures.

12. The finger-mounted suture cutter of claim 11, further comprising:
a cutting block, mounted to or part of the second arm,
wherein when the pivoting head pivots along the axis of the hinged portion, the cutting blade comes into contact with the cutting block, cutting a suture placed between the blade and cutting block.

13. The finger mounted suture cutter of claim 12, wherein the first arm include an adjustable finger mount to accommodate different finger diameters.

14. The finger mounted suture cutter of claim 3, wherein the second arm include an adjustable finger mount to accommodate different finger diameters.

15. The finger mounted suture cutter of claim 14, wherein the adjustable finger mounts of the first arm and the second arm comprise flexible portions of the first arm and second arm having sufficient spring tension to attach to the first and second portions of a human finger so as to securely clip the finger mounted suture cutter to the first and section portions of the human finger and to accommodate different finger diameters.

16. The finger mounted suture cutter of claim 11, wherein the cutting blade is at least partially enclosed by the pivoting head so as to prevent the inadvertent cutting of surrounding objects or material and a portion of the sharp edge remains exposed to allow cutting by drawing the edge against the suture to be cut.

17. The finger mounted suture cutter of claim 12, wherein when the pivoting head pivots along the axis of the hinged portion, the cutting blade comes into contact with the cutting block to cut sutures.

18. The finger mounted suture cutter of claim 11, wherein the first hinge portion comprises a live hinge of continuous flexible material, continuous with substantially all of the rest of the finger mounted suture cutter in order to reduce assembly steps needed for manufacture.

19. The finger mounted suture cutter of claim 11, wherein the a pivoting head, is coupled to the second arm through a second hinge portion, such that when the human finger is flexed, the second arm pulls the pivoting head toward the human finger.

20. The finger mounted suture cutter of claim 19, wherein the second hinge portion comprises a live hinge of continuous flexible material, continuous with substantially all of the rest of the finger mounted suture cutter in order to reduce assembly steps needed for manufacture.
